(19) United States
(12) Patent Application Publication
(10) Pub. No.: US 2026/0363986 A1
(43) Pub. Date: Nov. 19, 2026

(54) HAIR-PROTECTING COMPOSITION AND HAIR CARE PRODUCT COMPRISING SAME

(71) Applicants: MODAMODA CO., LTD., Seoul (KR); KOREA NATIONAL UNIVERSITY OF TRANSPORTATION INDUSTRY-ACADEMIC COOPERATION FOUNDATION, Chungju-si (KR)

(72) Inventors: Sung Young PARK, Chungju-si (KR); Seul Gi KIM, Cheongju-si (KR); Hyun Jeong WON, Seoul (KR)

(73) Assignees: MODAMODA CO., LTD., Seoul (KR); KOREA NATIONAL UNIVERSITY OF TRANSPORTATION INDUSTRY-ACADEMIC COOPERATION FOUNDATION, Chungju-si (KR)

(21) Appl. No.: 19/488,543

(22) PCT Filed: Jun. 1, 2023
(86) PCT No.: PCT/KR2023/007526

§ 371 Date: Nov. 26, 2025

(30) Foreign Application Priority Data
May 31, 2023 (KR) 10-2023-0070395

(51) Int. Cl. A61K 8/60; A61K 8/19; A61K 8/365; A61K 8/368; A61Q 5/00; A61Q 17/04

(52) U.S. Cl. CPC A61K 8/602; A61K 8/19; A61K 8/365; A61K 8/368; A61Q 5/002; A61Q 5/004; A61Q 17/04; A61K 2800/522; A61K 2800/591

(57) ABSTRACT

The present invention relates to: a hair-protecting composition comprising a polyphenol complex made of two or more types of polyphenol compounds in a solvent; and a hair care product comprising same. The hair-protecting composition of the present invention has UV blocking properties, conductivity, and durability and thus, when coated on hair, has the effects of preventing static electricity, repairing and ameliorating hair damage, and increasing mechanical strength, thereby ameliorating the issue of hair breakage.

### TECHNICAL FIELD

The present disclosure relates to a hair-protecting composition comprising a polyphenol complex formed from two or more kinds of polyphenol compounds in a solvent. The present disclosure also relates to a hair care product comprising the hair-protecting composition.

### BACKGROUND

Hair, once exposed to the external environment, is a human tissue that does not naturally regenerate. Due to physical and chemical factors such as permanent waving and dyeing, as well as environmental factors, hair is frequently damaged. In particular, when a hair iron or hair dryer is used every day, hair is often damaged by high-temperature heat styling tools. Wet hair is in a swollen state due to moisture, and when pressure is applied at a high temperature in this state, moisture evaporates and cracks are generated in the cuticle, cortex and medulla, thereby damaging the hair.

Various hair products have been developed in order to prevent such hair damage. For example, compositions using natural substances rather than synthetic raw materials, compositions using silk proteins, and compositions using protein hydrolysate raw materials such as keratin and collagen have been developed for hair protection. However, there have been limitations in protecting hair using only conventional hair-protecting products.

Accordingly, there is an urgent need to develop a novel hair composition capable of effectively regenerating damaged hair and enhancing the protective function of hair.

In view of this, the present inventors have developed a composition capable of coating hair to protect the hair, restoring damaged hair, and further preventing hair damage.

### PRIOR ART DOCUMENT

### Non-Patent Literature 0001

Ahn, H. J.; Lee, W.-S., “An Ultrastructural Study of Hair Fiber Damage and Restoration Following Treatment with Permanent Hair Dye,” Int. J. Dermatol. 2002, 41, 88-92.

### DISCLOSURE

### Technical Problem

An aspect of the present disclosure is to provide a hair-protecting composition which can effectively block ultraviolet rays, has high electrical conductivity to prevent the generation of static electricity in hair, remains stably coated on hair even after repeated washing to impart a lasting hair-protecting effect, imparts smoothness to hair damaged by frequent dyeing and bleaching, restores the hair cuticle layer, improves hair breakage, and exhibits excellent effects such as antioxidant activity.

Another aspect of the present disclosure is to provide a hair care product comprising the hair-protecting composition.

### Technical Solution

In order to achieve the above goals, the present disclosure provides a hair-protecting composition comprising a polyphenol complex formed from two or more kinds of polyphenol compounds in a solvent.

The present disclosure also provides a hair-protecting composition in which the polyphenol compounds comprise catechin, tannic acid, gallic acid, resveratrol, quercetin, protocatechuic acid, p-hydroxybenzoic acid, vanillic acid, caffeic acid, ferulic acid, luteolinidin, apigeninidin, and luteolin.

The present disclosure also provides a hair-protecting composition in which the polyphenol compounds comprise at least two kinds selected from tannic acid, gallic acid and caffeic acid.

The present disclosure also provides a hair-protecting composition comprising, based on 100 parts by weight of the solvent, 1.5 to 150 parts by weight of tannic acid, 0.5 to 10 parts by weight of gallic acid, and 0.01 to 0.1 parts by weight of caffeic acid.

The present disclosure also provides a hair-protecting composition comprising, based on 100 parts by weight of the solvent, 10 to 50 parts by weight of tannic acid, 1 to 5 parts by weight of gallic acid, and 0.05 to 0.1 parts by weight of caffeic acid.

The present disclosure also provides a hair-protecting composition in which the solvent has a pH in the range of 7 to 9.

The present disclosure also provides a hair-protecting composition in which the solvent is water or a phosphate buffer.

The present disclosure also provides a hair-protecting composition further comprising a conductive compound.

The present disclosure also provides a hair-protecting composition in which the conductive compound is graphene or graphene oxide.

The present disclosure also provides a hair-protecting composition comprising, based on 100 parts by weight of the solvent, 0.001 to 0.1 parts by weight of the conductive compound.

The present disclosure also provides a hair care product comprising the hair-protecting composition.

The present disclosure also provides a hair care product in which the hair-protecting composition is included in an amount of 0.5 to 10 wt % based on the total weight of the hair care product.

The present disclosure also provides a hair care product formulated in the form of a liquid, cream, lotion or gel.

The present disclosure also provides a hair care product which is a shampoo, rinse, treatment, scalp tonic, wax, spray, mousse, hair lotion, hair pack, hair essence, hair cream or hair dye.

### Advantageous Effects

The hair-protecting composition of the present disclosure includes a polyphenol complex formed from two or more kinds of polyphenol compounds in a solvent, and therefore imparts UV-blocking characteristics and durability to hair, provides conductivity to prevent static electricity on hair, restores and improves hair damage, and increases mechanical strength to improve hair breakage. In addition, it exhibits antioxidant effects and coats the hair to prevent discoloration of dyed hair.

### DESCRIPTION OF DRAWINGS

FIG. 1 is a schematic diagram illustrating the form in which a polyphenol complex is present, the polyphenol complex being formed from polyphenol compounds, namely tannic acid, gallic acid, and caffeic acid, in a solvent.

FIG. 2 illustrates the UV-vis spectrum results for the hair-protecting composition.

FIG. 3 shows the experimental results for the UV-blocking characteristics of the hair-protecting composition.

FIG. 4 shows the evaluation results of the anti-static characteristics of the hair care product.

FIG. 5 shows scanning electron microscope images of: untreated hair (Sample 1), damaged hair treated with 6% hydrogen peroxide (Sample 2), and damaged hair treated with 9% hydrogen peroxide (Sample 3), after treatment with the shampoo of Example 29; compared with bleached hair treated with the shampoo of Comparative Example 8.

FIG. 6 shows confocal microscope images of the hair surfaces of Sample 1 and the control group.

FIGS. 7A to 7C show the tensile test evaluation results for hair treated with the hair care product.

FIG. 8 illustrates the antioxidant evaluation results for the hair-protecting composition and the hair care product.

### MODE FOR INVENTION

The embodiments or aspects of the present disclosure are provided for the purpose of illustrating the technical spirit of the present disclosure. The scope of rights according to the present disclosure is not limited to the embodiments or aspects set forth below or to the detailed descriptions thereof.

Unless otherwise defined, all technical and scientific terms used herein have the meanings generally understood by a person of ordinary skill in the art to which the present disclosure pertains. All terms used herein are selected for the purpose of more clearly describing the present disclosure and are not intended to limit the scope of rights of the present disclosure.

Expressions such as “including,” “comprising,” or “having,” as used in the present disclosure, shall be understood as open-ended terms implying the possibility of including other embodiments unless otherwise stated in the phrase or sentence in which such expression appears.

Expressions such as “consisting of,” as used in the present disclosure, shall be understood as closed-ended terms excluding the possibility of including components other than the stated components.

Singular forms used in the present disclosure may include plural meanings unless otherwise specified, and the same applies to singular expressions used in the claims.

In one aspect of the present disclosure, the term “about” is used with the intention of including manufacturing tolerances within the specific numerical values or slight numerical adjustments within the technical scope of the present disclosure. For example, the term “about” may refer to a range of ±10%, ±5% in one aspect, or ±2% in another aspect, relative to the stated value. For the field of the present disclosure, such levels of approximation are appropriate unless a narrower range is specifically required.

Below, a detailed description will be given of the present disclosure.

One aspect of the present disclosure relates to a hair-protecting composition comprising a polyphenol complex formed from two or more types of polyphenol compounds in a solvent. The present disclosure pertains to the use of a polyphenol complex composition formed from two or more types of polyphenol compounds in a solvent as a hair-protecting agent. The present disclosure also relates to the use of a polyphenol complex composition formed from two or more types of polyphenol compounds in a solvent as a hair protector.

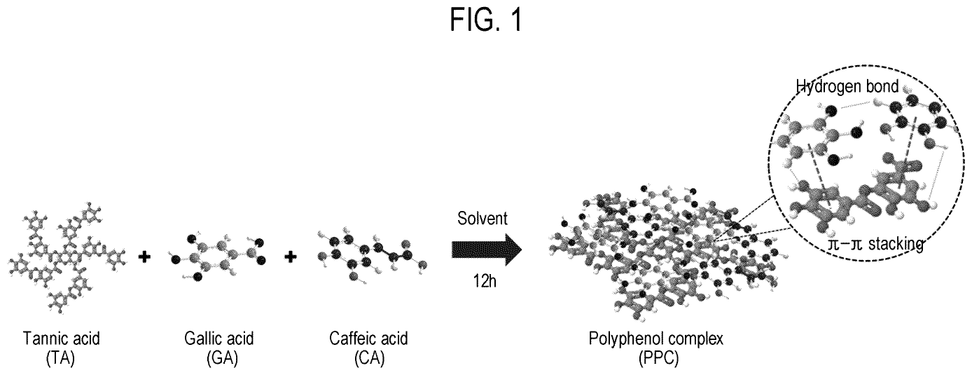

It has not been known that a polyphenol complex composition formed from two or more types of polyphenol compounds in a solvent exhibits excellent protective effects on hair. The inventors of the present disclosure discovered that, in a solvent, the two or more types of polyphenol compounds form a polyphenol complex through hydrogen bonding and π-π stacking between phenolic groups. In a solvent, the two or more types of polyphenol compounds become closely packed, increasing hydrogen bonding and thus forming a polyphenol complex (FIG. 1). The inventors further discovered that, when hair is treated with a hair product containing the polyphenol complex composition formed in a solvent, the hair obtains coating stability, high UV-blocking capability, conductivity, and durability, and that the polyphenol complex coats the hair to prevent static electricity, restore and improve hair damage, and increase mechanical strength to reduce hair breakage. The inventors also discovered that the polyphenol complex provides antioxidant effects and prevents discoloration of dyed hair by coating the hair. That is, the inventors found that the polyphenol complex has a hair-protecting function and can be usefully employed as a hair-protecting agent.

It has been known that polyphenol compounds may be used for hair dyeing. In such cases, the polyphenol compounds are included together with iron salts in hair dyes, and it is known that polyphenols and iron salts form coordination complexes via coordinate bonding. The inventors of the present disclosure surprisingly discovered that, unlike hair dyes containing iron salts, when two or more types of polyphenol compounds are included in a solvent, a polyphenol complex is formed through hydrogen bonding and π-π stacking between the different polyphenol compounds, and that such a polyphenol complex provides excellent hair-protecting effects. The polyphenol complex of the present disclosure is different in bonding type from complexes formed in the presence of iron salts. The hair-protecting composition comprising the polyphenol complex of the present disclosure may be substantially free of iron.

In one aspect of the present disclosure, the polyphenol compounds that form the polyphenol complex include, but are not limited to, catechin, tannic acid, gallic acid, resveratrol, quercetin, protocatechuic acid, p-hydroxybenzoic acid, vanillic acid, caffeic acid, ferulic acid, luteolinidin, apigeninidin, and luteolin.

In one aspect of the present disclosure, the hair-protecting composition comprises a polyphenol complex formed from two or more polyphenol compounds selected from tannic acid, gallic acid, and caffeic acid in a solvent.

In one aspect of the present disclosure, the hair-protecting composition comprises a polyphenol complex formed from polyphenol compounds including tannic acid, gallic acid, and caffeic acid in a solvent.

In one aspect of the present disclosure, the hair-protecting composition comprises a polyphenol complex formed from two or more polyphenol compounds selected from the group consisting of tannic acid, gallic acid, and caffeic acid in a solvent.

In one aspect of the present disclosure, the hair-protecting composition comprises a polyphenol complex formed from polyphenol compounds of tannic acid, gallic acid, and caffeic acid in a solvent.

Tannic acid has a high UV absorbance and can therefore provide UV protection, and it is rich in surface functional groups and stabilizes the molecular network, thereby improving the electrical conductivity of the composition. Gallic acid improves durability, reduces brittleness and flexibility issues, and exhibits plasticizing properties that enhance elasticity. Caffeic acid prevents lipid and protein oxidation and exhibits an excellent antioxidant effect by scavenging reactive oxygen species.

In addition, caffeic acid has a UV absorption wavelength range of 260 nm to 365 nm, which differs from the UV absorption wavelength range of 240 nm to 320 nm for tannic acid and gallic acid. Therefore, a hair-protecting composition containing tannic acid, gallic acid, and caffeic acid exhibits UV-blocking effects over a broad wavelength range.

In an embodiment, per 100 parts by weight of the solvent, each of the two or more polyphenol compounds may be included in an amount of 0.001 to 200 parts by weight, 0.001 to 100 parts by weight, 0.01 to 100 parts by weight, 0.1 to 100 parts by weight, 1 to 100 parts by weight, 0.001 to 50 parts by weight, 0.01 to 50 parts by weight, 0.1 to 50 parts by weight, 1 to 50 parts by weight, 0.001 to 20 parts by weight, 0.01 to 20 parts by weight, 0.1 to 20 parts by weight, 1 to 20 parts by weight, 0.001 to 10 parts by weight, 0.01 to 10 parts by weight, 0.1 to 10 parts by weight, or 1 to 10 parts by weight, but is not limited thereto.

In an embodiment, per 100 parts by weight of the solvent, tannic acid may be included in an amount of 0.1 to 200 parts by weight, 1 to 200 parts by weight, 1.5 to 150 parts by weight, 1.5 to 50 parts by weight, 5 to 100 parts by weight, 5 to 50 parts by weight, 10 to 30 parts by weight, or 10 to 20 parts by weight, but is not limited thereto.

In an embodiment, per 100 parts by weight of the solvent, gallic acid may be included in an amount of 0.01 to 100 parts by weight, 0.05 to 50 parts by weight, 0.1 to 50 parts by weight, 0.5 to 20 parts by weight, 0.5 to 10 parts by weight, 0.5 to 5 parts by weight, 0.5 to 3 parts by weight, or 1 to 3 parts by weight, but is not limited thereto.

In an embodiment, per 100 parts by weight of the solvent, caffeic acid may be included in an amount of 0.001 to 5 parts by weight, 0.001 to 1 part by weight, 0.001 to 0.5 part by weight, 0.01 to 0.1 part by weight, 0.02 to 0.08 part by weight, 0.03 to 0.07 part by weight, or 0.05 to 0.07 part by weight, but with no limitations thereto.

In an embodiment of the present disclosure, the hair-protecting composition comprises a polyphenol complex formed by including, per 100 parts by weight of the solvent, 1.5 to 150 parts by weight of tannic acid, 0.5 to 10 parts by weight of gallic acid, and 0.01 to 0.1 part by weight of caffeic acid.

In an embodiment of the present disclosure, the hair-protecting composition comprises a polyphenol complex formed by including, per 100 parts by weight of the solvent, 10 to 50 parts by weight of tannic acid, 1 to 5 parts by weight of gallic acid, and 0.05 to 0.1 part by weight of caffeic acid.

In an embodiment of the present disclosure, the hair-protecting composition comprises a polyphenol complex formed by including, per 100 parts by weight of the solvent, 10 to 50 parts by weight of tannic acid, 1 to 5 parts by weight of gallic acid, and 0.05 to 0.1 part by weight of caffeic acid.

In an embodiment of the present disclosure, the hair-protecting composition comprises a polyphenol complex formed by including, per 100 parts by weight of the solvent, 10 to 20 parts by weight of tannic acid, 1 to 3 parts by weight of gallic acid, and 0.05 to 0.08 part by weight of caffeic acid.

In an embodiment, the pH of the solvent is 1 to 13, 2 to 10, or 7 to 9.

In an embodiment, the solvent included in the hair-protecting composition has a pH of about 8.5.

In an embodiment, the solvent included in the hair-protecting composition is not particularly limited as long as two or more types of polyphenol compounds can form a polyphenol complex in the solvent, and may be water, purified water, distilled water, saline, or a phosphate buffer solution.

In an embodiment, a pH adjuster may additionally be included to adjust the pH of the solvent.

In an embodiment, the pH adjuster is not particularly limited as long as it is a material commonly used in the art, and one or more selected from hydrochloric acid aqueous solution, sodium hydroxide, potassium hydroxide, triethanolamine, citric acid, and sodium citrate may be used.

in an embodiment of the present disclosure, the hair-protecting composition further comprises a conductive compound.

In an embodiment, the conductive compound is graphene or graphene oxide.

In an embodiment, per 100 parts by weight of the solvent, the conductive compound may be included in an amount of 0.001 to 0.1 part by weight, 0.001 to 0.05 part by weight, or 0.005 to 0.01 part by weight, but is not limited thereto.

In an embodiment of the present disclosure, the polyphenol complex has adhesion and adsorption properties to hair, is attached to and coats the hair, and remains on the surface of the hair even after several washings.

In an embodiment of the present disclosure, the polyphenol complex has UV-blocking characteristics, conductivity, durability, and/or antioxidant effects, and thus coats the hair, prevents static electricity, restores and improves hair damage, and increases mechanical strength to improve hair breakage.

In an embodiment of the present disclosure, the polyphenol complex has hair-protecting and hair-repairing functions, and thus can be used for hair-protection purposes and/or hair-repairing purposes.

In an embodiment of the present disclosure, the polyphenol complex has hair-protecting and hair-repairing functions and therefore can be used as a hair protector.

In an embodiment of the present disclosure, two or more types of polyphenol compounds and a solvent may be introduced into a reactor and stirred to prepare the polyphenol complex.

In an embodiment, the reactor is not particularly limited as long as it is commonly used in the art.

In an embodiment of the present disclosure, the solvent and two or more types of polyphenol compounds are introduced into a reactor, stirred at 40 °C to 80 °C for 1 to 20 minutes at 100 to 300 rpm, and then stirred at room temperature for 10 to 20 hours to prepare the polyphenol complex.

In an embodiment of the present disclosure, the solvent and two or more types of polyphenol compounds are introduced into a reactor, stirred at 50 °C to 70 °C for 1 to 20 minutes at 100 to 300 rpm, and then stirred at room temperature for 10 to 50 hours to prepare the polyphenol complex.

In an embodiment, the solvent and two or more types of polyphenol compounds are introduced into a reactor, stirred at about 60 °C for about 5 minutes at about 100 to about 300 rpm, and then stirred at room temperature for about 12 hours to prepare the polyphenol complex.

In an embodiment of the present disclosure, the solvent and two or more types of polyphenol compounds are introduced into a reactor, stirred at 60 °C for 5 minutes at 100 to 300 rpm, and then stirred at room temperature for 12 hours to prepare the polyphenol complex.

In an embodiment of the present disclosure, the hair care product comprises the above-described hair-protecting composition. The hair care product of the present disclosure may be prepared by mixing the hair-protecting composition comprising the polyphenol complex formed by stirring two or more polyphenol compounds in a solvent, as described above, with hair care ingredients corresponding to each use (for example, a hair shampoo base or a hair treatment base).

In an embodiment, the hair-protecting composition may be included in the hair care product in an amount of 0.1 to 50 wt %, 0.1 to 20 wt %, 0.5 to 10 wt %, 1 to 10 wt %, or 1 to 5 wt %, based on the total weight of the hair care product, but is not limited thereto.

In an embodiment, the hair care product may be a conventional hair care product composition comprising the above-described hair-protecting composition, and the conventional hair care product composition may be, but is not limited to, shampoo, rinse, treatment, scalp tonic, wax, spray, mousse, hair lotion, hair pack, hair essence, hair cream, or a hair dye.

In an embodiment, the hair care product may be formulated as a liquid, cream, lotion, or gel.

In an embodiment, when the hair care product is formulated as a liquid, a solvent, solubilizer, or emulsifier may be used as a base ingredient, and the base ingredient may be, for example, water, ethanol, isopropanol, ethyl carbonate, ethyl acetate, benzyl alcohol, benzyl benzoate, propylene glycol, 1,3-butylene glycol oil, glycerol fatty acid ester, polyethylene glycol, or a fatty acid ester of sorbitan.

In an embodiment, when the hair care product is formulated as a cream, lotion, or gel, the base ingredient may include animal fibers, plant fibers, wax, paraffin, starch, tragacanth, cellulose derivatives, polyethylene glycol, silicone, bentonite, silica, talc, or zinc oxide.

In an embodiment, the hair care product may be shampoo, rinse, treatment, scalp tonic, wax, spray, mousse, hair lotion, hair pack, hair essence, hair cream, or a hair dye, but is not limited thereto.

In an embodiment, the hair care product may further comprise one or more selected from a surfactant, pH adjuster, swelling agent, buffer, preservative, fragrance, humectant, viscosity modifier, aliphatic alcohol, and vitamin, but is not limited thereto.

The surfactant may be any commonly used in the art without particular limitation, and examples thereof include cationic surfactants, anionic surfactants, nonionic surfactants, amphoteric surfactants, or mixtures thereof, and may be appropriately selected from surfactants known in the art so as to achieve the effects of the present disclosure.

More specifically, the surfactant may be an alkyl sulfate (AS), alkyl ether sulfate (AES), sodium alkane sulfonate (SAS), alkyl trimethyl ammonium halide, alkyl pyridinium halide, alkyl imidazolium halide, polyethylene oxide-based surfactant, polyvinyl-based water-soluble polymer, or the like. Non-limiting examples include sodium dodecyl sulfate (SDS), sodium octylbenzene sulfonate (NaOBS), sodium dodecylbenzene sulfonate (SDBS), ammonium lauryl sulfate (ALS), Triton X-100, and polyvinylpyrrolidone (PVP), but are not limited thereto.

The pH adjuster is not particularly limited as long as it is commonly used in the art to adjust the pH of hair care products, and one or more selected from sodium hydroxide, potassium hydroxide, triethanolamine, citric acid, and sodium citrate may be used.

The swelling agent is not particularly limited as long as it is commonly used in the art, and one or more selected from urea, thiourea, urea peroxide, polyoxymethylene urea, hydroxyethyl urea, ammonia, and salts thereof may be used.

The buffer is not particularly limited as long as it is commonly used in the art, and one or more selected from salicylic acid, sodium salicylate, lactic acid, and sodium lactate may be used.

The preservative may be any commonly used in the art without particular limitation, and examples include methylparaben, propylparaben, phenoxyethanol, methyl p-hydroxybenzoate, and ethyl p-hydroxybenzoate. The fragrance may be any commonly used in the art without particular limitation, and may be selected from natural or synthetic fragrances.

The humectant may be any commonly used in the art without particular limitation, and may include commonly used ingredients including glycols such as polyethylene glycol and oils.

The viscosity modifier may be any commonly used in the art without particular limitation, and may include one or more selected from poly stearate and sodium carboxymethyl cellulose. The aliphatic alcohol may be any commonly used in the art without particular limitation, and one or more aliphatic alcohols having 16 to 22 carbon atoms, such as cetyl alcohol, stearyl alcohol, or behenyl alcohol, may be used. The vitamin may be used to supplement nutrition to the hair.

In an embodiment, the hair care product may further include water, in addition to the above-described components, and the water may be purified water such as ion-exchanged water or distilled water. The content of water is not particularly limited, as long as it is an amount sufficient to dissolve or disperse the above-described components.

An aspect of the present disclosure includes a method of using the hair care product, the method comprising applying the hair care product to hair. When the hair care product is applied to the hair, the polyphenol complex formed from two or more polyphenol compounds included in the hair care product adheres to and coats the hair. The polyphenol complex included in the hair care product coats the hair and remains on the hair surface even after several washes, thereby protecting and repairing the hair.

### MODE FOR INVENTION

The apparatus ~Hereinafter, embodiments of the present disclosure will be described in detail. However, the following embodiments are provided only to facilitate understanding of the present disclosure, and the scope of the present disclosure is not limited thereto.

### Preparation of Hair-Protecting Composition

### Examples 1 to 21 and Comparative Examples 1 to 7

Distilled water (1 mL) was introduced into a reactor, and tannic acid, gallic acid, and/or caffeic acid in the amounts (unit: g) shown in Table 1 below were added. After stirring at 60 °C for 5 minutes at 100 to 300 rpm and then stirring at room temperature for 12 hours, hair-protecting compositions of Comparative Examples 1 to 7 and Examples 1 to 8 were prepared.

One milliliter of a solvent whose pH had been adjusted as shown in Table 1 below was prepared by adding 0.1 N HCl or 0.1 N NaOH to 1×PBS (phosphate buffered saline). Tannic acid, gallic acid, caffeic acid, and/or graphene oxide in the amounts (unit: g) shown in Table 1 below were then added, followed by stirring at 60 °C for 5 minutes at 100 to 300 rpm and subsequent stirring at room temperature for 12 hours to prepare hair-protecting compositions of Examples 9 to 21.

TABLE 1

| | Polyphenol Compound | | | Conductive | |
|---|---|---|---|---|---|
| | Tannic | Gallic | Caffeic | Compound | |
| | acid | acid | acid | Graphene | |
| Composition | (g) | (g) | (g) | oxide (g) | Solvent |
| C. Ex. 1 | 0.015 | — | — | — | Distilled |
| C. Ex. 2 | 0.15 | — | — | — | water |
| C. Ex. 3 | 1.5 | — | — | — | |
| C. Ex. 4 | — | 0.005 | — | — | |
| C. Ex. 5 | — | 0.02 | — | — | |
| C. Ex. 6 | — | 0.1 | — | — | |
| C. Ex. 7 | — | — | 0.0006 | — | |
| Ex. 1 | 0.15 | 0.02 | — | — | |
| Ex. 2 | 0.15 | — | 0.0006 | — | |
| Ex. 3 | — | 0.02 | 0.0006 | — | |
| Ex. 4 | 0.15 | 0.005 | 0.0006 | — | |
| Ex. 5 | 0.15 | 0.02 | 0.0006 | — | |
| Ex. 6 | 0.15 | 0.1 | 0.0006 | — | |
| Ex. 7 | 0.015 | 0.02 | 0.0006 | — | |
| Ex. 8 | 1.5 | 0.02 | 0.0006 | — | |
| Ex. 9 | 0.15 | 0.02 | 0.0006 | — | pH 1 |
| Ex. 10 | 0.15 | 0.02 | 0.0006 | — | pH 3 |
| Ex. 11 | 0.15 | 0.02 | 0.0006 | — | pH 5 |
| Ex. 12 | 0.15 | 0.02 | 0.0006 | — | pH 7.4 |
| Ex. 13 | 0.15 | 0.02 | 0.0006 | — | pH 8.5 |
| Ex. 14 | 0.15 | 0.02 | 0.0006 | — | pH 11 |
| Ex. 15 | 0.15 | 0.02 | 0.0006 | — | pH 13 |
| Ex. 16 | 0.15 | 0.02 | 0.0006 | 0.00001 | pH 8.5 |
| Ex. 17 | 0.15 | 0.02 | 0.0006 | 0.0001 | pH 8.5 |
| Ex. 18 | 0.15 | 0.02 | 0.0006 | 0.001 | pH 8.5 |
| Ex. 19 | 0.15 | 0.02 | — | — | pH 8.5 |
| Ex. 20 | 0.15 | — | 0.0006 | — | pH 8.5 |
| Ex. 21 | — | 0.02 | 0.0006 | — | pH 8.5 |

### Preparation of Hair Care Products Containing Hair-Protecting Composition

### Examples 22 to 30 and Comparative Examples 8 to 11

Either 1 mL of distilled water introduced into the reactor or 1 mL of a solvent whose pH had been adjusted as shown in Table 2 below by adding 0.1 N HCl or 0.1 N NaOH to 1×PBS (phosphate buffered saline) was added as a solvent to the reactor. Thereafter, tannic acid, gallic acid, caffeic acid, and/or graphene oxide in the amounts (unit: g) shown in Table 2 below were introduced. The mixture was stirred at 60 °C for 5 minutes at 100 to 300 rpm, followed by stirring at room temperature for 12 hours to prepare the hair-protecting composition.

Subsequently, 30 g of a hair shampoo base (Modamoda, Republic of Korea) was added to the above composition as an additional ingredient for shampoo formulation, and the mixture was stirred at 50 °C at 50 to 100 rpm for 5 minutes to prepare a hair-protecting shampoo.

### Comparative Example 12

A shampoo was prepared by introducing 30 g of a hair shampoo base (Modamoda, Republic of Korea) into a reactor, adding 0.15 g of tannic acid, 0.02 g of gallic acid, and 0.0006 g of caffeic acid, and stirring at 50 °C at 50 to 100 rpm for 5 minutes.

### Comparative Example 13

A shampoo was prepared in the same manner as in Example 22 except that 1 mL of a solvent whose pH had been adjusted to 8.5 by adding 0.1 N NaOH to 1×PBS (phosphate buffered saline) was used in the reactor, and 0.15 g of tannic acid, 0.02 g of gallic acid, 0.0006 g of caffeic acid, and 0.001 g of FeSO₄ were added.

### Example 31

After adding tannic acid, gallic acid, and caffeic acid in the amounts shown in Table 2 below, a hair-protecting composition was prepared in the same manner as described in Example 22.

Thereafter, 30 g of a hair treatment base (Modamoda, Republic of Korea) was added to the above composition as an additional ingredient for hair-treatment formulation, and the mixture was stirred at 50 °C at 50 to 100 rpm for 5 minutes to prepare a hair-protecting treatment.

### Example 32

After adding tannic acid, gallic acid, and caffeic acid in the amounts (unit: g) shown in Table 2 below, a hair-protecting composition was prepared in the same manner as in Example 22.

Subsequently, 30 g of a hair tonic base (Modamoda, Republic of Korea) was added to the above composition as an additional ingredient for scalp-tonic formulation, and the mixture was stirred at 50 °C at 50 to 100 rpm for 5 minutes to prepare a hair-protecting scalp tonic.

TABLE 2

| | Polyphenol compound | | | Conductive compound | | |
|---|---|---|---|---|---|---|
| | Tannic acid | Gallic acid | Caffeic acid | Graphene oxide | | |
| Composition | (g) | (g) | (g) | (g) | Solvent | Base |
| C. Ex. 8 | — | — | — | — | Distilled | Shampoo |
| C. Ex. 9 | 0.15 | — | — | — | water | |
| C. Ex. 10 | — | 0.02 | — | — | | |
| C. Ex. 11 | — | — | 0.0006 | — | | |
| Ex. 22 | 0.15 | 0.02 | — | — | | |
| Ex. 23 | 0.15 | — | 0.0006 | — | | |
| Ex. 24 | — | 0.02 | 0.0006 | — | | |
| Ex. 25 | 0.15 | 0.02 | 0.0006 | — | | |
| Ex. 26 | 0.15 | 0.02 | — | — | pH 8.5 | |
| Ex. 27 | 0.15 | — | 0.0006 | — | | |
| Ex. 28 | — | 0.02 | 0.0006 | — | | |
| Ex. 29 | 0.15 | 0.02 | 0.0006 | — | | |
| Ex. 30 | 0.15 | 0.02 | 0.0006 | 0.00001 | | |
| Ex. 31 | 0.15 | 0.02 | 0.0006 | — | | Treatment |
| Ex. 32 | 0.15 | 0.02 | 0.0006 | — | | Scalp tonic |

### Experimental Example 1. Evaluation of Optical Properties of Hair-Protecting Composition

### (1) UV-Vis Spectrum of the Hair-Protecting Composition

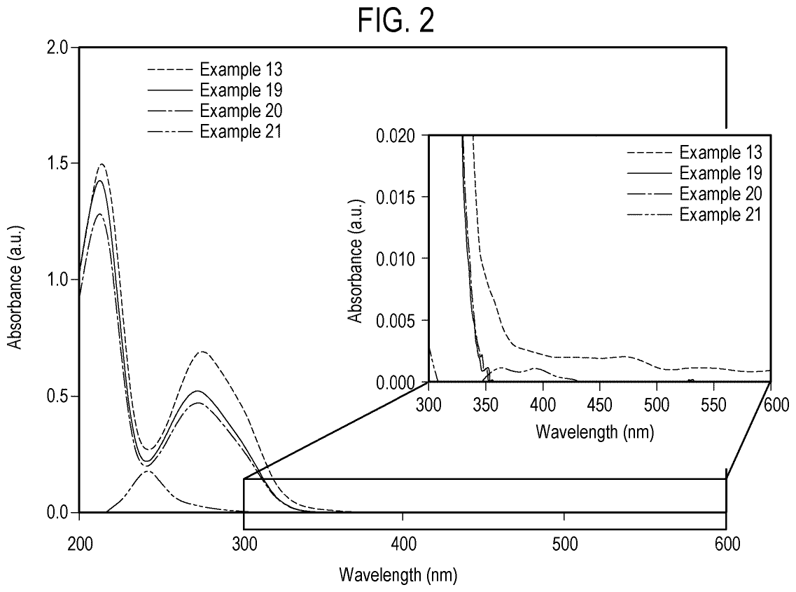

The compositions of Examples 13 and 19 to 21 were subjected to UV-vis spectral analysis using a UV-vis spectrophotometer (Ubi-490, Micro Digital), and the UV-vis spectra were obtained (FIG. 2). Referring to FIG. 2, it was confirmed that the hair-protecting compositions of the present disclosure absorb UV light. In particular, in the case of the composition of Example 13, which contains tannic acid, gallic acid, and caffeic acid, UV absorption was observed in the range of 380 nm to 600 nm, confirming that the composition absorbs light over a broad spectrum.

### (2) Evaluation of UV-Blocking Properties of Hair-Protecting Composition

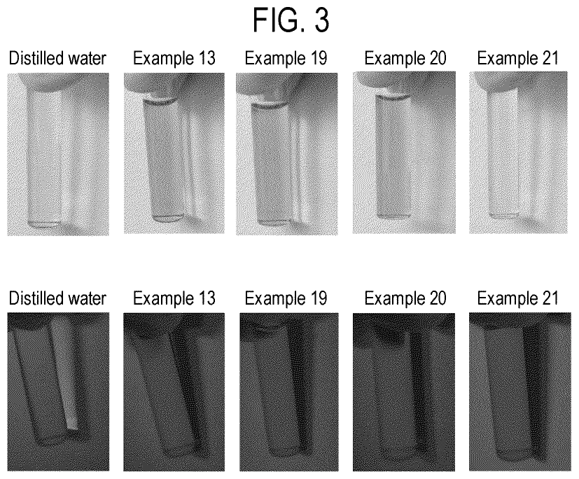

The compositions of Examples 13 and 19 to 21 were injected into glass cuvettes, and UV transmittance was evaluated by observing whether UV light penetrated through to a paper screen. Distilled water injected into a glass cuvette was used as a control sample. The experimental results are shown in FIG. 3.

Referring to FIG. 3, UV light was visible on the paper screen in the case of the control sample containing distilled water, indicating that UV was transmitted. In contrast, for Examples 13 and 19 to 21, UV light was not observed on the paper screen, confirming that UV was not transmitted.

### Experimental Example 2. Confirmation of FT-IR Spectrum of Hair-Protecting Composition

The compositions of Examples 1 to 3, 5, 13, 16, and 19 to 21, and those of Comparative Examples 2, 5, and 7 were analyzed using FT-IR (Madison, Thermo Scientific), and the hydroxyl group peaks in the FT-IR spectra were confirmed. The hydroxyl group peaks for each composition are shown in Table 3 below.

TABLE 3

| | Position of peak of hydroxy group |
|---|---|
| Composition | (OH, cm⁻¹) |
| C. Ex. 2 | 3400 |
| C. Ex. 5 | 3400 |
| C. Ex. 7 | 3400 |
| Ex. 1 | 3495 |
| Ex. 2 | 3495 |
| Ex. 3 | 3405 |
| Ex. 5 | 3524 |
| Ex. 13 | 3544 |
| Ex. 16 | 3531 |
| Ex. 19 | 3496 |
| Ex. 20 | 3495 |
| Ex. 21 | 3414 |

Examples 1 to 3, 5, 13, 16, and 19 to 21 exhibited a greater shift in the hydroxyl peak compared with Comparative Examples 2, 6, and 7. In Comparative Examples 2, 5, and 7, the hydroxyl peak did not shift because hydrogen bonding was not strong. However, in Examples 1 to 3, 5, 13, 16, and 19 to 21, two or more polyphenols were included, and these compounds were closely packed with one another, resulting in increased hydrogen bonding, which is reflected in the observed shift of the hydroxyl peak.

### Experimental Example 3. Conductivity and Surface Contact Angle of Hair-Protective Composition

The hair-protective compositions of Examples 1 to 21 and Comparative Examples 1 to 7 were dip-coated onto a silicon wafer (Si wafer) substrate at room temperature for 24 hours. After dip-coating, the substrates were washed with distilled water and dried to obtain substrates coated with the hair-protective compositions. The conductivity of the hair-protective compositions before and after 10 wash cycles was measured using a source meter (Keithley, 2450). Additionally, the surface contact angle of the coated substrates before and after 10 wash cycles was measured using a contact-angle analyzer (Contact angle, DO3210, Kruss). Each measurement was performed three times, and the average values are shown in Table 4 below.

TABLE 4

| | Resistance(MΩ/cm²) | | Surface Contact Angle (°) | |
|---|---|---|---|---|
| | Before | After | Before | After |
| C. Ex. 1 | 29.6 | 29.7 | 10.3 | 10.4 |
| C. Ex. 2 | 25.4 | 25.8 | 7.2 | 7.2 |
| C. Ex. 3 | 21.5 | 21.5 | 6.5 | 6.4 |
| C. Ex. 4 | 35.4 | 35.7 | 35.6 | 35.6 |
| C. Ex. 5 | 28.9 | 28.9 | 32.2 | 32.3 |
| C. Ex. 6 | 33.2 | 33.4 | 30.2 | 30.2 |
| C. Ex. 7 | 26.1 | 26.2 | 72.7 | 72.8 |
| Ex. 1 | 4.32 | 4.32 | 11.3 | 11.3 |
| Ex. 2 | 4.5 | 4.5 | 12.5 | 12.6 |
| Ex. 3 | 6.3 | 6.5 | 40.5 | 40.5 |
| Ex. 4 | 16.4 | 16.5 | 14.3 | 14.3 |
| Ex. 5 | 3.53 | 3.55 | 12.3 | 12.4 |
| Ex. 6 | 3.76 | 3.79 | 11.1 | 11.1 |
| Ex. 7 | 6.34 | 6.35 | 24.6 | 24.6 |
| Ex. 8 | 9.54 | 9.54 | 9.8 | 9.8 |
| Ex. 9 | 4.54 | 4.64 | 17.5 | 17.6 |
| Ex. 10 | 4.65 | 4.70 | 16.7 | 16.8 |
| Ex. 11 | 3.43 | 3.44 | 14.3 | 14.4 |
| Ex. 12 | 1.65 | 1.65 | 12.7 | 12.9 |
| Ex. 13 | 0.71 | 0.75 | 10.4 | 10.5 |
| Ex. 14 | 1.28 | 1.34 | 10.6 | 11.6 |
| Ex. 15 | 1.26 | 1.35 | 10.8 | 12.8 |
| Ex. 16 | 0.55 | 3.43 | 10.7 | 10.5 |
| Ex. 17 | 0.39 | 3.54 | 20.5 | 12.7 |
| Ex. 18 | 0.34 | 3.77 | 30.6 | 18.4 |
| Ex. 19 | 1.30 | 1.38 | 10.3 | 10.2 |
| Ex. 20 | 1.10 | 1.21 | 11.4 | 11.6 |
| Ex. 21 | 2.40 | 2.61 | 40.4 | 40.5 |

It was confirmed that Examples 1 to 21 exhibited lower resistance compared with Comparative Examples 1 to 7. Examples 1 to 21 exhibited higher conductivity than Comparative Examples 1 to 7, indicating that the compositions effectively dissipate electrostatic charge on hair and exhibit excellent anti-static performance. In particular, Examples 13 and 19 to 21 showed higher conductivity than Examples 5 and 1 to 3. This is attributed to ions present in the PBS solvent imparting ionic conductivity to the polyphenol components.

In Examples 1 to 21, even after 10 washes with distilled water, the resistance and surface contact angle of the coated surfaces showed almost no change. This demonstrates that the hair-protective compositions of the present disclosure remain stably coated on the surface even after repeated washing.

Examples 16 to 18 exhibited changes in conductivity after washing, which is considered to be due to the graphene oxide additionally included as the conductive compound in these examples. It appears that the non-adhesive graphene oxide detached after 10 wash cycles, resulting in changes in conductivity and increased hydrophilicity, thereby decreasing the surface contact angle.

### Experimental Example 4. Evaluation of Anti-Static Properties of Hair Care Products

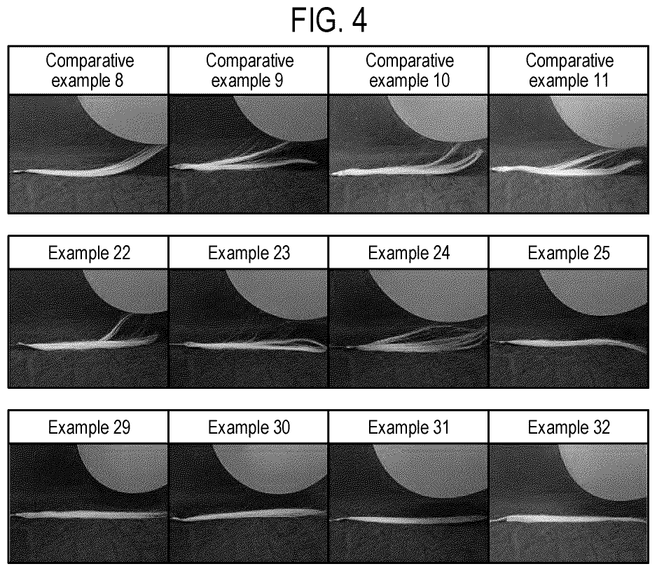

Bleached hair samples (Yeoshin, Republic of Korea) were coated with each of the hair care products according to Examples 22 to 25 and 29 to 32 and Comparative Examples 8 to 11 for 30 minutes. The bleached hair samples were washed with water, incubated at room temperature for 1 hour, and then dried using a dryer. To evaluate anti-static properties, a rubber balloon was inflated with air, rubbed three times with a cotton fabric, and then placed near the hair. The results are shown in FIG. 4.

As shown in FIG. 4, Comparative Examples 8 to 11 exhibited static electricity, with hair being attracted to the balloon. Examples 22 to 24 showed a reduced amount of hair being attracted to the balloon compared with Comparative Examples 8 to 11, confirming their anti-static properties.

Examples 25 and 29 to 32 showed no hair adhering to the balloon, confirming excellent anti-static performance. Since the hair-protective composition of the present disclosure exhibits conductivity, hair care products containing the hair-protective composition demonstrate anti-static effects.

### Experimental Example 5. Evaluation of Hair Recovery and Improvement by Hair Care Products

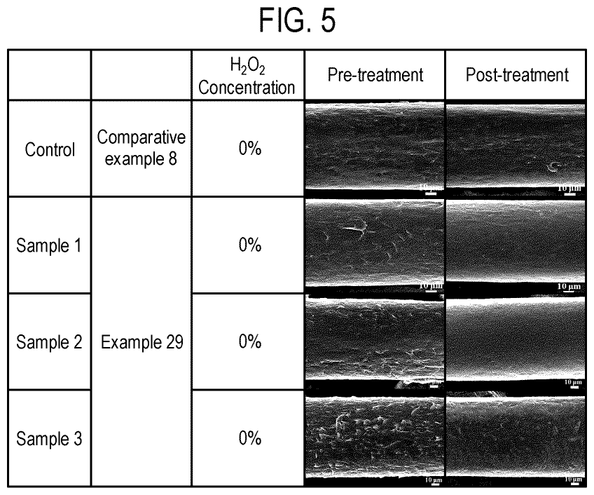

When hair is dyed or bleached, oxidizing agents such as hydrogen peroxide (H₂O₂) are included, which damage the hair. In this experimental example, 6% and 9% hydrogen peroxide were applied to bleached hair samples for 12 hours to prepare damaged hair. The shampoo of Example 29 was applied to undamaged hair (Sample 1), hair treated with 6% hydrogen peroxide (Sample 2), and hair treated with 9% hydrogen peroxide (Sample 3), followed by washing and drying. As a control, bleached hair samples were washed and dried using the shampoo of Comparative Example 8, which did not contain polyphenol compounds. The surfaces of Samples 1 to 3 and the control hair were examined by scanning electron microscopy (SEM) (JSM-6700F, JEOL, Akishima, Tokyo, Japan) (FIG. 5).

As shown in FIG. 5, the degree of cuticle damage in Samples 1 to 3 increased according to the hydrogen peroxide treatment level (0%, 6%, 9%). After treatment with the hair care product of Example 29, the surfaces of Samples 1 to 3 appeared smoother, confirming that the hair care product of the present disclosure restored the damaged hair cuticle layer. In contrast, the control sample in FIG. 5 showed no change in cuticle condition before and after shampooing.

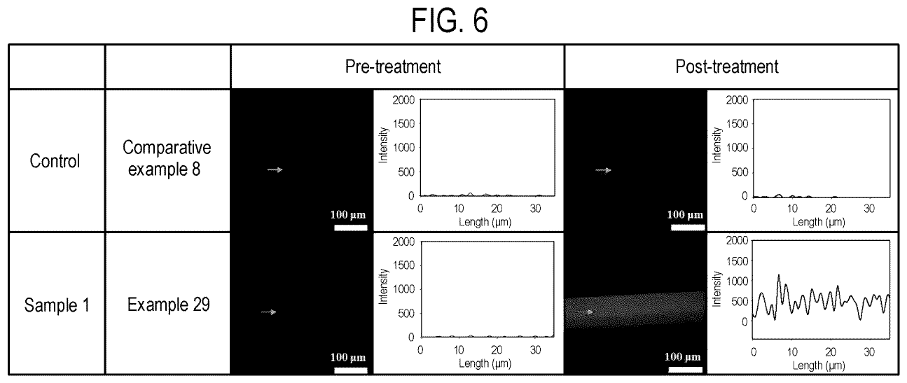

Additionally, the surfaces of Sample 1 and the control were examined using an ECLIPSE Ti2-E confocal microscope (Nikon, Tokyo, Japan) to observe coating on the hair surface (FIG. 6). As shown in FIG. 6, the control sample exhibited no fluorescence even after shampoo application. In contrast, hair treated with the hair care product of the present disclosure exhibited blue fluorescence, demonstrating that the hair-protective composition was coated across the entire hair surface. These results confirm that the hair care product of the present disclosure coats the hair and restores and improves damaged hair.

### Experimental Example 6. Evaluation of Improvement in Hair Breakage by Hair Care Products

When hair is dyed or bleached, oxidizing agents such as hydrogen peroxide (H₂O₂) are included, which damage the hair. In this experimental example, 6% and 9% hydrogen peroxide were applied to bleached hair samples for 12 hours to prepare damaged hair. The shampoo of Example 29, which is a hair care product according to the present disclosure, was applied to undamaged hair (Sample 1), hair treated with 6% hydrogen peroxide (Sample 2), and hair treated with 9% hydrogen peroxide (Sample 3), followed by washing and drying. As controls, bleached hair samples were washed and dried using the shampoo of Comparative Example 8, which did not contain polyphenol compounds. Additionally, bleached hair samples were washed and dried using the shampoos of Comparative Examples 12 and 13, respectively.

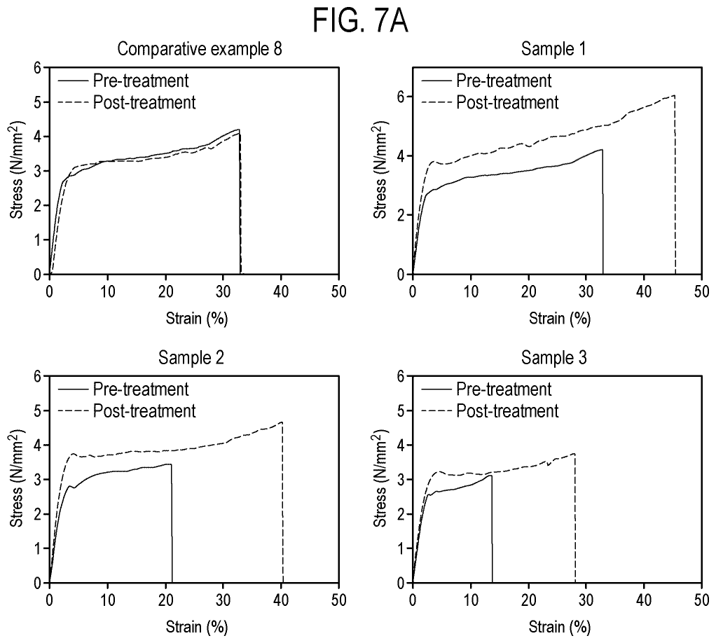

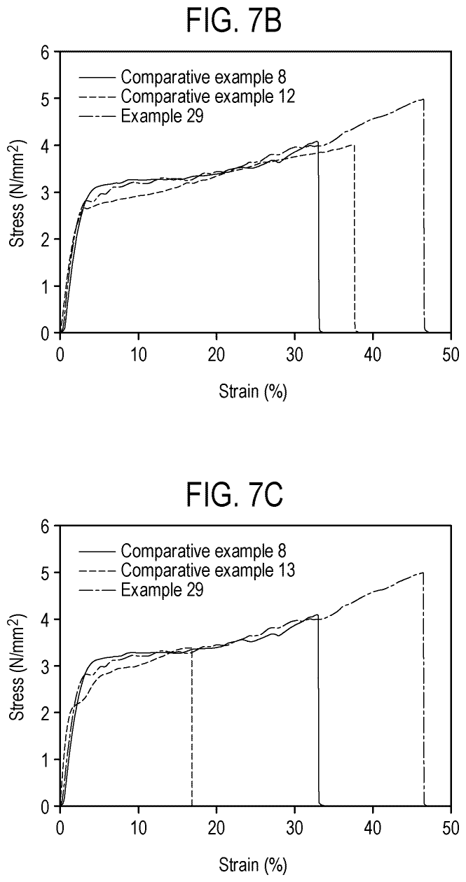

The samples treated with the hair care product of Example 29 (Samples 1 to 3) and the hair samples of Comparative Examples 8 and 12 to 13 were subjected to tensile testing using a universal testing machine (SurTA Extra 1A system, Chemilab Co., Republic of Korea) (FIGS. 7a to 7c). The tensile stress and strain values of Samples 1 to 3 of Example 29 and the control of Comparative Example 8 are shown in Table 5 below.

TABLE 5

| | | | Before Treatment | | After Treatment | |
|---|---|---|---|---|---|---|
| | Hair | H₂O₂ | Tensile | | Tensile | |
| | Care | Concen- | Stress | Strain | Stress | Strain |
| | Product | tration | (N/mm²) | (%) | (N/mm²) | (%) |
| Control | C. Ex. 8 | 0% | 4.21 | 32.75 | 4.08 | |
| Sample 1 | Ex. 29 | 0% | 4.21 | 33.02 | 6.04 | |
| Sample 2 | | 6% | 3.44 | 20.97 | 4.64 | |
| Sample 3 | | 9% | 3.05 | 13.50 | 3.79 | |

As shown in Table 5, Samples 1 to 3 treated with the hair care product of the present disclosure exhibited increases in both tensile stress and strain after treatment compared to before treatment. In contrast, the control showed almost no change in tensile stress or strain before and after treatment. This confirms that the hair care product of the present disclosure restores the mechanical strength of hair and improves hair breakage.

Example 29 is a hair care product manufactured by mixing a shampoo base with a hair-protective composition containing a polyphenol complex formed from solvent, tannic acid, gallic acid, and caffeic acid. Comparative Example 12 is a hair care product manufactured by directly adding tannic acid, gallic acid, and caffeic acid into the shampoo base. As shown in FIG. 7b, Comparative Example 12 exhibited a significantly lower strain value compared with Example 29, confirming that no polyphenol complex was formed in Comparative Example 12, resulting in reduced mechanical strength.

Comparative Example 13 is a hair care product prepared by adding into the shampoo base a composition containing a coordination complex formed from tannic acid, gallic acid, caffeic acid, and an iron salt. As shown in FIG. 7c, Comparative Example 13 exhibited a significantly lower strain value compared with Example 29, and even significantly lower than the shampoo base alone (Comparative Example 8). It is therefore considered that when a coordination complex formed from a polyphenol compound and an iron salt is used, hair breakage becomes worse, rendering it unsuitable for use as a hair-protective agent.

### Experimental Example 7. Antioxidant Evaluation of Hair-Protective Composition and Hair Care Products

A 0.2 mM aqueous 2,2-diphenyl-1-picrylhydrazyl (DPPH) solution was mixed with the hair-protective composition of Example 13 at a 1:1 ratio, shielded from light, and stirred at 37 °C for 30 minutes. The DPPH inhibition rate was calculated by comparing the absorbance peak at 517 nm using UV-vis spectroscopy. The equation for calculating the DPPH inhibition rate is as follows.

DPPH inhibition rate (%)=(A_{std}−Aₛₐₘ)/A_{std}×100%
wherein, A_{std} is the absorbance of the DPPH standard and Aₛₐₘ is the absorbance of the sample.

The antioxidant evaluation was also performed in the same manner for the hair care products of Comparative Example 8 and Example 29, and the DPPH inhibition rates were calculated.

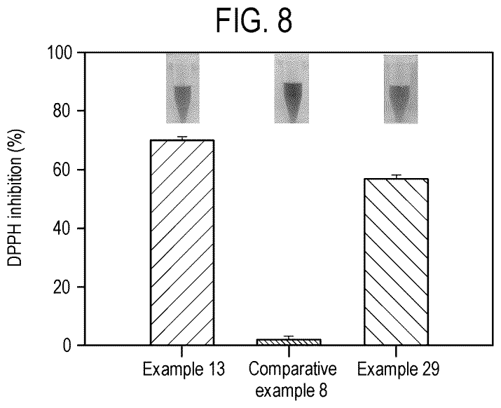

The calculated DPPH inhibition rates were 69% for the hair-protective composition of Example 13, nearly 0% for the hair care product of Comparative Example 8, and 58% for the hair care product of Example 29 (FIG. 8).

These results confirm that the hair-protective composition of the present disclosure and the hair care products containing the same exhibit antioxidant effects. In particular, Comparative Example 8, which did not contain polyphenol compounds, exhibited nearly 0% DPPH inhibition. Thus, the antioxidant effect of the hair care product of the present disclosure arises from the inclusion of the hair-protective composition of the present disclosure.

### Experimental Example 8. Evaluation of Prevention of Discoloration of Dyed Hair Using Hair Care Products

Hair samples dyed red were treated and dried with each of the hair care products of Examples 29, 31, and 32 and Comparative Example 8. For each hair sample, the color difference values (L, a) were measured before UV (302 nm) irradiation and after 7 days following irradiation using a colorimeter (KONICA MINOLTA, Japan). The measured values are shown in Table 6 below.

TABLE 6

| | L value | | a value | |
|---|---|---|---|---|
| | 0 hour | After 7 days | 0 hour | After 7 days |
| C. Ex. 8 | 54.3 | 56.3 | 9.8 | 7.2 |
| Ex. 29 | 54.3 | 54.1 | 9.6 | 9.5 |
| Ex. 31 | 54.4 | 54.0 | 9.7 | 9.7 |
| Ex. 32 | 54.2 | 53.8 | 9.5 | 9.7 |

As shown in Table 6, the dyed hair treated with Examples 29, 31, and 32 exhibited almost no difference in color difference values (L, a) between before irradiation and after 7 days following irradiation, compared with the dyed hair treated with Comparative Example 8. The dyed hair treated with Examples 29, 31, and 32 did not undergo discoloration due to the UV-blocking effect of the hair-protective composition coated on the hair.

From the above description, those skilled in the art to which the present disclosure pertains will understand that the present disclosure can be implemented in various specific forms without departing from the technical spirit or essential characteristics of the present disclosure. Accordingly, the embodiments described above are to be understood as illustrative rather than limiting in all respects. The scope of the present disclosure should be interpreted as encompassing all modifications and variations derived from the meaning and scope of the claims set forth below and the equivalents thereof, rather than being limited by the foregoing detailed description.

1. A hair-protective composition comprising a polyphenol complex formed from two or more polyphenol compounds in a solvent.

2. The hair-protective composition of claim 1, wherein the polyphenol compounds are selected from two or more of the group consisting of catechin, tannic acid, gallic acid, resveratrol, quercetin, protocatechuic acid, p-hydroxybenzoic acid, vanillic acid, caffeic acid, ferulic acid, luteolinidin, apigenin, and luteolin.

3. The hair-protective composition of claim 1, wherein the polyphenol compounds comprise two or more selected from tannic acid, gallic acid, and caffeic acid.

4. The hair-protective composition of claim 1, wherein the polyphenol compounds comprise tannic acid, gallic acid, and caffeic acid.

5. The hair-protective composition of claim 3, wherein the hair-protective composition comprises 1.5 to 150 parts by weight of tannic acid, 0.5 to 10 parts by weight of gallic acid, and 0.01 to 0.1 parts by weight of caffeic acid, per 100 parts by weight of the solvent.

6. The hair-protective composition of claim 3, wherein, the hair-protective composition comprises 10 to 50 parts by weight of tannic acid, 1 to 5 parts by weight of gallic acid, and 0.05 to 0.1 parts by weight of caffeic acid, per 100 parts by weight of the solvent.

7. The hair-protective composition of claim 1, wherein the solvent has a pH of 7 to 9.

8. The hair-protective composition of claim 1, wherein the solvent is water or a phosphate buffer.

9. The hair-protective composition of claim 1, further comprising a conductive compound.

10. The hair-protective composition of claim 9, wherein the conductive compound is graphene or graphene oxide.

11. The hair-protective composition of claim 9, wherein the conductive compound is contained in an amount of 0.001 to 0.1 parts by weight, based on 100 parts by weight of the solvent

12. A hair care product comprising the hair-protective composition of claim 1.

13. The hair care product of claim 12, wherein the hair-protective composition is contained in an amount of 0.5 to 10 wt % based on the total weight of the hair care product.

14. The hair care product of claim 12, wherein the product is formulated as a liquid, cream, lotion, or gel.

15. The hair care product of claim 12, wherein the product is a shampoo, rinse, treatment, scalp tonic, wax, spray, mousse, hair lotion, hair pack, hair essence, hair cream, or hair dye.
